(19) United States
(12) Patent Application Publication
(10) Pub. No.: US 2022/0218585 A1
(43) Pub. Date: Jul. 14, 2022

(54) COLLAGEN COMPOSITIONS AND USES THEREOF

(71) Applicant: DATUM BIOTECH LTD., Lod (IL)

(72) Inventor: Thomas BAYER, Tel Aviv (IL)

(73) Assignee: DATUM BIOTECH LTD., Lod (IL)

(21) Appl. No.: 17/613,974

(22) PCT Filed: May 27, 2019
(86) PCT No.: PCT/IL2019/050597

§ 371 Date: Nov. 24, 2021

(51) Int. Cl. A61K 8/65; A61K 8/34; A61K 8/44; A61Q 19/08; A61K 38/39; A61K 47/10; A61K 47/18

(52) U.S. Cl. CPC A61K 8/65; A61K 8/345; A61K 8/44; A61K 47/183; A61K 38/39; A61K 47/10; A61Q 19/08

(57) ABSTRACT

The present invention provides compositions comprising collagen, including kits thereof and methods for their use.

### BACKGROUND OF THE INVENTION

Collagen is the main structural protein in the extracellular space in the various connective tissues in the body. As the main component of connective tissue, it is the most abundant protein in mammals, making 25% to 35% of the whole-body protein content. Collagen consists of amino acids wound together to form triple-helices of elongated fibrils. It is mostly found in fibrous tissues such as tendons, ligaments, and skin.

Molecular collagen has an approximate diameter of 1.6 nm with a length of about 300 nm. (Shu-Wei Changl and Markus J. Buehler, Materials Today 2014, 17, 2). Molecular collagen is only stable at acidic pH unless it is stabilized by surfactant (Dale Devore et al, Journal of Biomedical Materials Research A, 03/2016 Vol. 104A, 3).

Molecules larger than 500 Da are problematic to be delivered through the skin barrier. Research suggests that glycerol has a reversable effect on cells, tissue and particularly on the physical properties of collagen. This effect is used to enhance the penetration depth to visualize structures deeper inside the skin.

According to literature (Xiang Wen et al, J. Biophoton. 3, No. 1-2, 44-52 (2010)) it is believed that the interaction of glycerol within the upper layers of the skin causes a contraction and re-alignment of collagen fibers and fibrils, a contraction of cells and extracellular matrix.

### SUMMARY OF THE INVENTION

The present invention provides a composition comprising collagen, glycerol and EDTA, wherein said composition has a pH of at least 6.5.

In some embodiments, the pH of the composition is between 6.5 and 7.5. In other embodiments, the pH of the composition is 6.5, 6.6, 6.7, 6.8, 6.9, 7.0, 7.1, 7.2, 7.3, 7.4, 7.5.

In some embodiments, the composition of the invention is a dermatological (topical) composition (i.e. penetrating only the close proximal area of skin where administration was provided, including the epidermis and dermis layers). Most often topical administration means application to body surfaces such as the skin or mucous membranes to treat ailments via a designated topical formulation. In some embodiments, said composition is epicutaneous, meaning that said composition is applied directly to the skin.

In other embodiments, a composition of the invention is a transdermal (including transcutaneous and percutaneous) composition (i.e. systemically penetrating through the area of skin where administration was provided).

In some embodiments, a composition of the invention further comprises at least one of buffer, preservative, vitamin, mineral, skin toning agent, skin moisturizing agent, and any combination thereof.

In some embodiments, collagen is at least 0.1% wt of the composition. In other embodiments, collagen is in the range of between 0.1-5% wt of the composition. In other embodiments, collagen is in the range of between 0.1-1% wt of the composition. In other embodiments, collagen is in the range of between 0.1-2% wt of the composition. In other embodiments, collagen is in the range of between 0.1-3% wt of the composition. In further embodiments, said collagen is 0.1, 0.2, 0.3, 0.4, 0.5, 1.0, 1.5, 2.0, 2.5, 3.0, 3.5, 4.0, 4.5, 5.0% wt of the composition

In some embodiments, glycerol is at least 1% wt of the composition. In further embodiments, glycerol is in the range of between 1-30% wt of the composition. In further embodiments, glycerol is in the range of between 5-25% wt of the composition. In further embodiments, glycerol is in the range of between 10-30% wt of the composition. In other embodiments, glycerol is 1, 2, 3, 4, 5, 6, 7, 8, 9, 10, 15, 20, 25, 30% wt of the composition.

In some embodiments, EDTA is at least 0.1% wt of the composition. In further embodiments, EDTA is in the range of between 0.1-2.5% wt of the composition. In further embodiments, EDTA is in the range of between 0.1-2.0% wt of the composition. In other embodiments, EDTA is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5% wt of the composition.

In further embodiments, a composition of the invention is in the form selected from an aqueous solution, oily solution, an oil-in-water solution, a gel, a foam, a cream, a suspension, a dispersion, an ointment, a lotion, a powder, a solid, vapor, paste, tincture and any combinations thereof.

In some embodiments, a composition of the invention is comprised in a device for dermal (topical) delivery of said composition. Such a device may include a patch, a mask, a bandage, a roller, tape, sponge, and any combinations thereof.

In some embodiments, a composition of the invention is comprised in a device for transdermal delivery of said composition. Such a device may include an injection, a patch, a mask, a bandage, a roller, tape, sponge, and any combinations thereof.

The invention further provides a kit comprising at least one composition as disclosed herein below and above and at least one means for dermal or transdermal delivery.

In some embodiments, a kit of the invention further comprises at least one further skin treating composition.

The present invention also relates to compositions in admixture with pharmaceutically acceptable auxiliaries, and optionally other active agents. The auxiliaries must be “acceptable” in the sense of being compatible with the other ingredients of the composition and not deleterious to the recipients thereof.

Pharmaceutical compositions include those suitable for topical (including dermal, transdermal, buccal and sublingual), vaginal or parenteral (including subcutaneous, and intradermal) administration or administration via an implant or a patch. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, anti-oxidants, and wetting agents.

The invention further includes a pharmaceutical composition, as hereinbefore described, in combination with packaging material, including instructions for the use of the composition for a use as hereinbefore described.

The compositions may be presented in unit-dose or multi-dose containers, for example sealed vials and ampoules, and may be stored in a freeze-dried (lyophilised) condition requiring only the addition of sterile liquid carrier, for example water, prior to use. For dermal or transdermal administration, includes, also aqueous and non-aqueous sterile injection, gels, patches or sprays.

The exact dose and regimen of administration of the composition will necessarily be dependent upon the effect to be achieved and may vary with the particular formula, the route of administration, and the age and condition of the individual subject to whom the composition is to be administered.

The invention further provides a composition as disclosed herein above and below, for use in the treatment of a condition of a subject benefiting from collagen such as wound healing, skin aging (including skin wrinkling, skin elasticity, skin hydration), dermal collagen density, bone density, osteoporosis, age-related skin conditions, age-related bone conditions, scleroderma including symptoms thereof and any combination thereof.

The invention further provides a composition as disclosed herein above and below, for use in the treatment of a condition of a skin of a subject.

When relating to the treatment of a condition of a skin of a subject, it should be understood to include any skin condition including, but not limited to wrinkling of skin (including susceptibility of skin to wrinkle), elasticity of skin, hydration of skin, reduction of cellulite appearance, acne (including spread and susceptibility of skin to develop acne), comedos (blackheads, including spread and susceptibility of skin to develop blackheads) scleroderma including symptoms thereof and any combinations thereof.

The invention further provides a method of treating a condition of a skin of a subject, comprising administering to said subject a composition as disclosed herein above and below.

In another one of its aspects the invention provides a composition comprising collagen and glycerol, wherein said composition has a pH of at least 6.5. In some embodiments, the composition has a pH of between 6.5 and 7.5.

In another aspect a composition comprising collagen and glycerol, wherein said composition has a pH of at least 6.5, is directed for use in stabilizing said collagen in a molecular form.

In another aspect a composition comprising collagen and glycerol, wherein said composition has a pH of at least 6.5, is directed, for use in preventing fibrillation of said collagen.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the UV/VIS spectra of purified collagen that was brought (9:1 collagen-fibrillation buffer) to neutral pH in the presents of 0, 2.5, 5, 7.5, 10% glycerol. UV/VIS spectra have been collected using a 1 cm cuvette.

FIG. 2 shows the UV/VIS spectra of purified collagen that was fibrillated (9:1 collagen-fibrillation buffer) at neutral pH, and wherein glycerol was subsequently added thereto in increasing amounts of 0, 2.5, 5, 7.5, 10, 12.5, 15 or 20% glycerol. UV/VIS spectra have been collected using a 1 cm cuvette.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

TABLE 1
An embodiment of a composition of the invention

| Wt % in composition | Component |
|---|---|
| 1.0% | PBS |
| 0.9% | EDTA |
| 20.1% | Glycerol |
| 0.2% | Collagen |
| 77.9% | water |

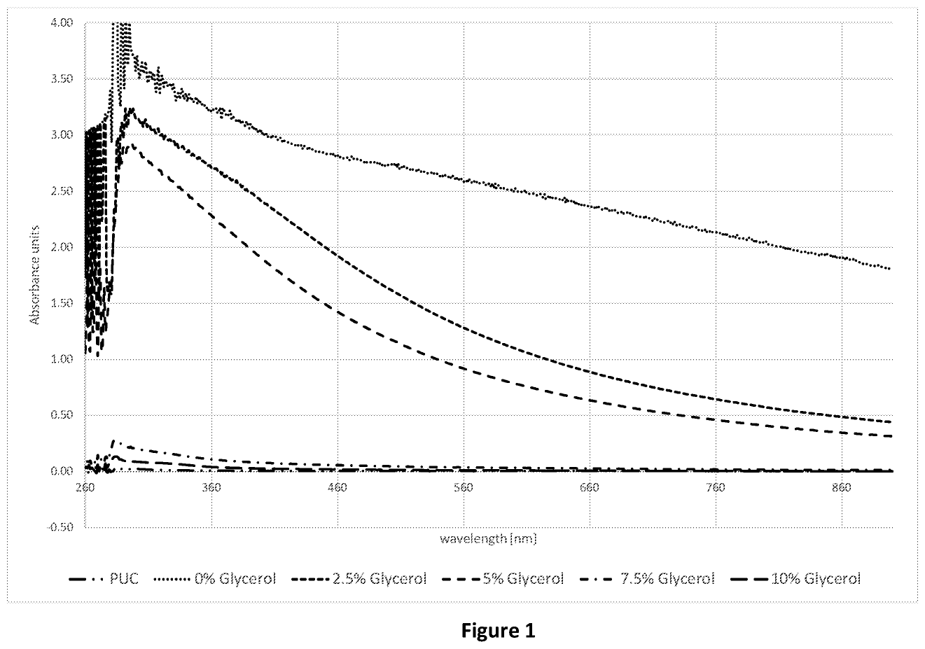

Example 1: Purified collagen was fibrillated (9:1 collagen-fibrillation buffer) at neutral pH in the presence (or absence) of 0, 2.5, 5, 7.5, 10% glycerol. Fibrillation was gradually inhibited with increasing concertation of glycerol. UV/VIS spectra have been collected using a 1 cm cuvette (See FIG. 1).

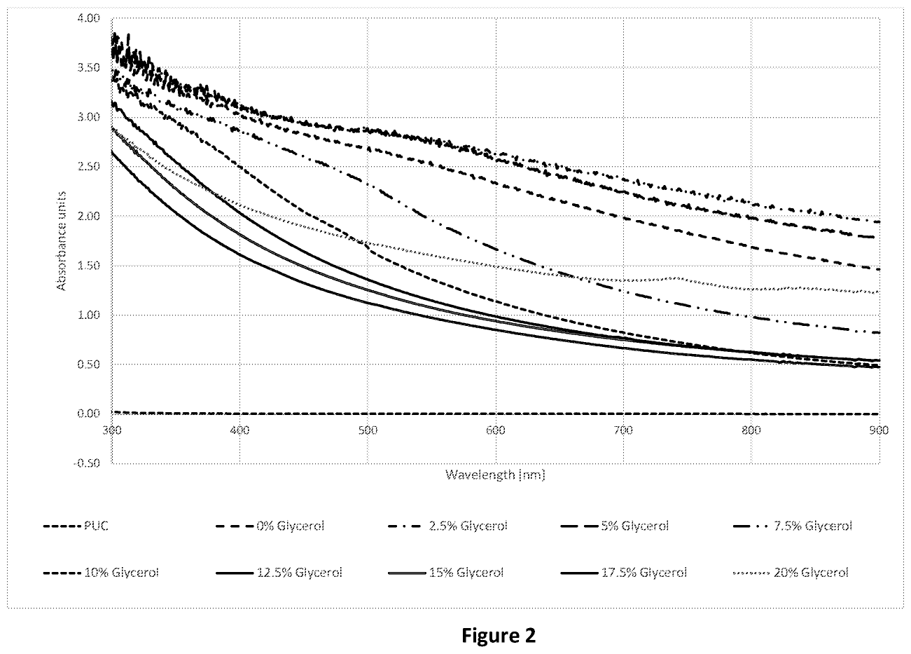

Example 2: Purified collagen was fibrillated (9:1 collagen-fibrillation buffer) at neutral pH. Subsequently, 0, 2.5, 5, 7.5, 10, 12.5, 15 or 20% glycerol was added to the fibrillated collagen. As can be seen from the UV/VIS spectra (see FIG. 2) fibrillation was gradually reversed with increasing concertation of glycerol, but not completely.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition comprising collagen, glycerol and EDTA, wherein said composition has a pH of at least 6.5.

2. A composition according to claim 1, having a pH of between 6.5 and 7.5.

3. A composition according to claim 1, being a dermatological composition.

4. A composition according to claim 1, being a transdermal composition.

5. A composition according to any one of the preceding claims, further comprising at least one of buffer, preservative, vitamin, mineral, skin toning agent, skin moisturizing agent, and any combination thereof.

6. A composition according to any one of the preceding claims, wherein collagen is at least 0.1% wt of the composition.

7. A composition according to any one of the preceding claims, wherein collagen is in the range of between 0.1-5% wt of the composition.

8. A composition according to any one of the preceding claims, wherein glycerol is at least 1% wt of the composition.

9. A composition according to any one of the preceding claims, wherein glycerol is in the range of between 1-30% wt of the composition

10. A composition according to any one of the preceding claims, wherein EDTA is at least 0.1% wt of the composition.

11. A composition according to any one of the preceding claims, wherein EDTA is in the range of between 0.1-2% wt of the composition.

12. A composition according to any one of the preceding claims, being in the form of an aqueous solution, oily solution, an oil-in-water solution, a gel, a foam, a cream, a suspension, a dispersion, an ointment, a lotion, a powder, a solid, vapor, paste, tincture and any combinations thereof.

13. A composition according to any one of the preceding claims, for use in the treatment of a condition of the skin of a subject.

14. A method of treating a condition of the skin of a subject, comprising administering to said subject a composition as disclosed in any of claims 1 to 13.

15. A kit comprising at least one composition according to any one of the preceding claims and at least one means for dermal or transdermal delivery.

16. A kit according to claim 16, further comprising at least one further skin treating composition.

17. A composition comprising collagen and glycerol, wherein said composition has a pH of at least 6.5.

18. A composition according to claim 17, having a pH of between 6.5 and 7.5.

19. A composition according to claim 17 or 18, wherein collagen is at least 0.1% wt of the composition.

20. A composition according to any one of claims 17 to 19, wherein collagen is in the range of between 0.1-5% wt of the composition.

21. A composition according to any one of claims 17 to 20, wherein glycerol is at least 1% wt of the composition.

22. A composition according to any one of claims 17 to 21, for use in stabilizing said collagen in a molecular form.

23. A composition according to any one of claims 17 to 21, for use in preventing fibrillation of said collagen.
